SEQUENCE LISTING (7 sequences)

SEQ ID NO: 1 - DNA, 336 bp, Synechococcus elongatus
atgtcaatgaagacgctccctaaagaaaggagatttgaaacgttttcatatctgccccctctctctgatagacaaatcgctgctcaaatcgaatatatgatcgaacaaggttttcatccattaatcgaatttaatgaacattctaatccagaagaattttattggactatgtggaagctccctctttttgattgtaaatctcctcaacaggttttagatgaagtgagagagtgtagatctgaatatggtgattgttatatcagagttgctggttttgataatatcaaacaatgtcaaactgtttcttttatcgttcatagacctggaagatactaa

SEQ ID NO: 2 - DNA, 1419 bp, Synechococcus elongatus
atgcctaagactcaatcggctgccggttacaaggcaggtgtaaaagattacaaactaacttattatactccagattatacacccaaagacactgacttactagccgcctttcgcttttcgccccagccaggtgttccagctgatgaagctggtgctgctatcgctgctgaatcttctactggtacttggactactgtttggactgatttattaactgatatggacagatacaaaggcaaatgttaccatattgaaccggttcaaggtgaggaaaattcttattttgcttttatcgcataccctctagatctttttgaagagggttctgttactaatatcttaacttctatcgtcggtaatgtctttggctttaaggccattcgtagcctacgtcttgaagacatcaggtttccagttgctttagttaaaacttttcaaggtccaccacatggtatccaagtagaacgggatcttttaaataaatatggcagaccgatgctcgggtgcacgattaagccgaagctcgggctttctgctaaaaattatggtagagctgtttatgaatgtttacgtggtggtttagattttactaaagatgatgaaaatatcaattctcaaccgttccagcgttggcgggaccgattcctctttgtggccgacgcgatccataaatctcaagctgaaactggtgaaatcaaaggtcattatttaaatgtaacggcgcctacatgtgaagaaatgatgaagcgagcagaatttgctaaggaactaggtatgcctatcatcatgcatgattttttaactgctggttttactgctaatactactttagctaaatggtgccgggacaatggagtcctattacatatccatcgtgccatgcacgcggtcattgatcgtcaaaggaatcatggtatccattttagagttttagctaaatgtttaagattatctggtggtgatcatttacattctggtactgtcgtgggaaagcttgagggtgacaaggcatctacattaggttttgttgatttaatgagagaagatcatatcgaagctgatagatctagaggtgttttttttactcaagactgggcgtcgatgccgggggtgctcccagttgcttctggtggtatccatgtttggcacatgccggcgttagttgaaatctttggtgatgattctgttttacaatttggtggtggtactttaggtcatccatggggtaatgcaccaggtgctactgctaatagagttgctttagaagcttgtgttcaagctagaaatgaaggtagagatttatatagagagggtggtgatattttaagggaagcaggtaaatggtcgcctgaactggcagccgccctcgatttatggaaagaaatcaaatttgaatttgaaactatggataaattataa

SEQ ID NO: 3 - DNA, 486 bp, Synechococcus elongatus
atgcaatttatgggtactgcttctaggatggcgtcgacgcaacgggccaagcctatggagatgccgaggattagccgtgatactgctagaatgttagttaattatttaacttatcaagctgtttgtgttatcagagatcaattagctgaaactaatccagctggtgcctatagattacaagttttttctgctgaattttcttttcaagatggtgaagcttatttagctgctttattaaatcatgatagagaattaggactaagggtgatgacggtaagggaacatttagctgaacatattctagattatcttccagaaatgacgattgcccaaattcaagaggccaacattaaccatagaagagcacttttagaaaggttaacaggccttggggctgagccatctttaccggaaacggaggtctcagacagaccctcagattctgctactccagatgatgcttctaatgcttctcatgctgattaa

SEQ ID NO: 4 - DNA, 1002 bp, Synechococcus elongatus
atgtctaaaccagatagagttgttttaatcggtgttgctggtgattctggttgtggtaaatctacatttcttaacaggttagctgatttatttggtactgaattaatgactgttatttgtttagatgattatcattcgttagatcgtaaaggcagaaaggaagcgggtgtaactgctttagatcctagagctaataattttgatttaatgtatgaacaagttaaagctttaaaaaatggtgaaactatcatgaaaccaatctataatcatgaaactggtttaatcgatccacctgaaaagatcgagccaaacagaattattgtaattgaagggttacacccactttatgacgaacgagttcgcgaacttttagatttttctgtttatttagatatcgatgatgaagttaaaatcgcttggaaaatccaaagagatatggccgaaagaggtcattcttatgaagatgttttagcctcaattgaggctagaaggccagattttaaagcatatattgaaccgcaacggggacacgctgatatcgttattcgtgtaatgcccactcaacttatcccgaatgacactgagaggaaagtcctaagggtacaattaatccagagagaaggaagggatggatttgaaccagcttatttatttgatgaaggttctacaattcaatggacgccttgtggcagaaagttaacatgtagctatcctggcattcgcttagcttatggtccagatacttattatggtcatgaagtttctgtccttgaagtggatggacaatttgaaaatttagaagaaatgatttacgttgaaggtcatttatctaaaactgatactcaatattatggtgaattaactcatctacttttacaacacaaagattatccaggttctaataatggtactggtttattccaagtgctaacgggtctcaagatgcgggccgcctatgaaaggttaacttctcaagctgctccagttgctgcttctgtttaa

SEQ ID NO: 5 - DNA, 312 bp, Synechococcus elongatus
atggctgccgtctcattatctgtttctactgttactccattaggtgatagagtttttgttaaagttgctgaagctgaagaaaaaactgctggtggtatcatcttaccagataatgctaaagaaaaaccacaagtcggtgaaattgtcgctgttggtccaggtaaaagaaatgatgatggttcaagacaagctccagaagttaaaatcggtgataaagttttatattctaaatatgctggtactgatattaaattaggtaatgatgattatgttcttttatctgaaaaagatatcttagctgttgtcgcttaa

SEQ ID NO: 6 - DNA, 1668 bp, Synechococcus elongatus
atggctaaattaatcttatttcatgaagattcaagacaagcattagaaaggggtgttaatgctttagctaatgctgttaaagttactttaggtccaagaggtagaaatgttttattagaaaaaaaatttggtgctccagaaatcatcaatgatggtgtttctatcgctaaagaaatcgaattagaagatccacatgaaaatgcaggtgcaagactagttcaagaagttgctgctaaaactaaagaaatcgctggtgatggtactactactgctactgttttagctcaagctatcgttagagaaggtttaactaatgttgctgctggtgctaatccaatcgttttaagaagaggtatcgaaaaagctgttgctactttagttgaagctatcgctgctaaagctcaaccagttgctgatgaagctgctatcagatctatcgctgctgtttctgctggtaatgatgatgaagttggtcaaatgatcgctgatgctgttgctaaagttactaaagatggtgttatcacagttgaagaatctaaatctttagctactgaattagaagtcgttgaaggtatgcaatttgatagaggttatttatctccatattttgttactgatcaagatagacaagtagttgaatatgataatccattaatcttattaactgataaaaaaatcgcttctatccaagatttagttccagttttagaagatgttgctagagctggtagaccattattaatcatcgctgaagatatcgaaggtgaagctttagctactttagttgttaataaagctagaggtgttttaaatactgttgctgttaaagctccagcttttggtgatagaagaaaagctatcttacaagatatcgctgttttaactggtggtcaagttatctctgaagaagttggtttatctttagctgatgctaattcttctgttttaggtaaagctcaaaaaatcactatctctaaagatactactatcatcgttgctggtgatgaaaataaagctgatgttgctgctagaatcgctcaaatcagaagatctttagaagaaactgattctgattatgatagagaaaaattacaagaaagaatcgctaaattagctggtggtgttgctgttatcaaagttggtgctccaactgaaactgaattaaaaaatagaaaattaagaatcgaagatgctttaaatgctactagagctgctatcgaagaaggagttgttccaggtggtggtactactttattacatttagcttctgctttaacttctttacaagcttctttaactgttgctgatgaaaaattaggtgttgaaatcgttgctagagctttagaagctccattaagacaaattgctgataatgctggtgcagaaggttctgttgttgtcgaaaaattaagagataaagattttaattttggttataatgctttaactggtcaatatgaagatttagttgcttctggtatcttagatccagctaaagttgttagatctgctttacaagatgctgcttctgttgcttctttaatcttaactactgaagttttagttgttgatcaacctgaaccagaaccagctatgcctgctggtggtgatatgggtggtatgggtggtatgggtatgcctggtatgggtggtatgggtatgatgtaa

SEQ ID NO: 7 - DNA, 1635 bp, Synechococcus elongatus
atggctaaaagaatcatctataatgaaaatgctagaagagctttagaaaaaggtatcgatatcttagctgaagctgttgctgttactttaggtccaaaaggtagaaatgtcgtcttagaaaagaaatttggtgcaccacaaattatcaatgatggtgttactatcgctaaagaaatcgaattagaagatcatatcgaaaatactggtgttgctttaatcagacaagcagcttcaaaaacaaatgatgctgctggtgatggtactactactgctactgttttagctcatgctgttgtcaaagaaggtttaagaaatgttgctgctggtgctaatgctatcttattaaaaagaggtatcgataaagctacaaattttcttgtcgaacaaattaaatcacatgctcgtccagtcgaagattctaaatctatcgcacaagttggtgcaatctctgctggtaatgattttgaagttggtcaaatgatcgctgatgctatggataaagttggtaaagaaggtgttatctctttagaagaaggtaaatctatgactactgaattagaagttactgaaggtatgcgttttgataaaggttatatctctccatattttgctactgatactgaaagaatggaagccgtctttgatgaaccatttatcttaatcactgataaaaaaatcggattagttcaagatcttgtcccagttttagaacaagttgctagagctggtagaccattagttattatcgcagaagatatcgaaaaagaagctttagctactttagttgttaatagattaagaggtgtcttaaatgttgcagctgtcaaagctccaggttttggtgatagaagaaaagctatgttagaagatatcgctgttcttacaggtggtcaacttatcacagaagatgctggtttaaaattagatactactaaattagatcaattaggtaaagctagaagaatcactatcactaaagataatactactatcgttgctgaaggtaatgaagctgctgttaaagctagagtcgatcaaattagaaggcaaattgaagaaacagaaagctcttatgataaagaaaagttacaagaaagattagctaaattatctggtggtgtcgcagttgtcaaagttggtgctgctactgaaactgaaatgaaagatagaaaattaagattagaagatgctatcaatgctactaaagctgctgttgaagaaggtatcgttccaggtggtggtactactttagctcatttagctccacaattagaagaatgggcaactgctaatttatctggtgaagaattaactggtgctcaaatcgttgctagagctttaactgctccattaaaaagaatcgctgaaaatgctggtttaaatggtgctgttatctctgaaagagtcaaagaattaccatttgatgaaggttatgatgcatcaaataatcaatttgttaatatgtttactgctggtattgttgatccagctaaagttacaagatcagctttacaaaatgctgcttctatcgctgctatggttttaactactgaatgtatcgttgttgataaaccagaaccaaaagaaaaagctccagctggtgctggtggtggtatgggtgattttgattattaa

(19) United States
(12) Patent Application Publication
(10) Pub. No.: US 2018/0187204 A1
(43) Pub. Date: Jul. 5, 2018

(54) COMBINATION OF BACTERIAL CHAPERONES POSITIVELY AFFECTING THE PHYSIOLOGY OF A NATIVE OR ENGINEERED EUKARYOTIC CELL

(71) Applicants: Institut National de la Recherche Agronomique, Paris (FR); Institut National des Sciences Appliquees de Toulouse, Toulouse Cedex 4 (FR); Centre National de la Recherche Scientifique, Paris (FR)

(72) Inventors: Denis Pompon, Pechabou (FR); Stephane Guillouet, Vallegue (FR); Jillian Marc, Toulouse (FR); Nathalie Gorret, Vallegue (FR); Carine Bideaux, Le Vernet (FR); Christel Boutonnet, Pins Justaret (FR); Florence Bonnot, Saix (FR)

(21) Appl. No.: 15/542,682

(22) PCT Filed: Jan. 15, 2016
(86) PCT No.: PCT/EP2016/050832

§ 371 Date: Jul. 11, 2017

(30) Foreign Application Priority Data
Jan. 16, 2015 (EP) 15305048.9
Jan. 16, 2015 (EP) 15305049.7

(51) Int. Cl. C12N 15/81; C07K 14/245; C12P 21/02; C07K 14/395

(52) U.S. Cl. CPC C12N 15/81; C07K 14/395; C12P 21/02; C07K 14/245

(57) ABSTRACT

The invention relates to the expression of a specific combination of three bacterial chaperones in a eukaryotic cell, which significantly improves the growth properties thereof and the properties thereof relating to resistance to physicochemical stresses, especially when said cell comprises additional engineering using at least one non-native gene. A preferred combination of chaperones comprises the chaperones GroES and GroEL of E. coli and the chaperone RbcX of Synechococcus elongatus.

The present invention relates to the field of cellular engineering, in particular of eukaryotic cells. More particularly, the invention relates to eukaryotic cells having improved growth and/or metabolic properties, and to the use thereof for the production of compounds of interest. The invention relates in particular to eukaryotic cells expressing a specific combination of chaperones. The invention finds applications notably in the field of the production of recombinant proteins.

### Technological Background

There currently exist many expression systems for proteins of interest used in fields as varied as biotechnologies, food processing, the medicines industry or diagnostic research, fundamental and/or applied. Among these expression systems, eukaryotic cells, and yeasts in particular, play an important role notably because of their glycosylation capacity and their ease of culture in industrial conditions. These cells have limits, however, notably due to toxicity constraints related to, for example, the accumulation of products of interest (for example a high concentration of ethanol or other alcohol) or to direct or indirect toxicity of the proteins expressed or to the energy load which requires the presence of expression plasmids.

### SUMMARY OF THE INVENTION

The present invention provides eukaryotic cells having improved performance, enabling the development of optimized expression systems.

In the context of a project directed at introducing a synthetic Calvin cycle in yeast, the Inventors observed, surprisingly, that co-expression, in yeast, of a set of three bacterial chaperones (RbcX, GroES and GroEL) confer upon eukaryotic cells remarkable metabolic properties, notably in terms of expression and functional folding of heterologous or endogenous proteins. Pursuing their investigations, the Inventors also showed that this combination of chaperones also make it possible to accelerate the growth of yeasts and to remove the toxic effects of other engineering, for example when the kinase PRK is co-expressed in the cell. The Inventors further confirmed and obtained the same effects on performance on various eukaryotic cells, confirming the advantage and the great potential of these unexpected results.

An object of the present invention thus relates to a eukaryotic cell, characterized in that it expresses the chaperones RbcX, GroES and GroEL.

In a particular embodiment, the present invention relates to a transformed eukaryotic cell, characterized in that it contains:

(i) an expression cassette containing a sequence encoding a chaperone RbcX involved in the folding of a bacterial form I RuBisCO enzyme, under the transcriptional control of a suitable promoter;
(ii) an expression cassette containing a sequence encoding a bacterial general chaperone GroES under the transcriptional control of a suitable promoter; and
(iii) an expression cassette containing a sequence encoding a bacterial general chaperone GroEL under the transcriptional control of a suitable promoter.

The invention is also directed to a eukaryotic cell containing:

(i) a sequence encoding a chaperone RbcX under the transcriptional control of a suitable promoter;
(ii) a sequence encoding a chaperone GroES under the transcriptional control of a suitable promoter; and
(iii) a sequence encoding a chaperone GroEL under the transcriptional control of a suitable promoter.

According to a particular embodiment, the eukaryotic cells of the invention do not contain a sequence encoding the RbcL and/or RbcS subunit of a bacterial form I RuBisCO enzyme.

The invention proposes in particular a transformed yeast as indicated above.

The invention also has as an object the use of a combination of expression cassettes enabling the expression of a chaperone RbcX and the chaperones GroES and GroEL, to improve the physiology of a eukaryotic cell, and in particular to increase the growth rate of said eukaryotic cell and/or to increase the resistance of said eukaryotic cell to an environmental stress and/or to increase the resistance of said cell to the toxicity of a compound synthesized by the eukaryotic cell and/or to produce a recombinant protein.

The invention also has as an object a biotechnological process for producing at least one compound selected from chemical molecules and proteins, characterized in that it comprises a step of culturing a eukaryotic cell according to the invention under conditions enabling the synthesis, by said eukaryotic cell, of this compound, and a step of collecting said compound.

The invention proposes more particularly a process for producing a recombinant protein comprising (i) inserting a sequence encoding said protein into a eukaryotic cell expressing RbcX, GroES and GroEL, (ii) culturing said cell under conditions enabling the expression of said sequence and optionally (iii) collecting or purifying said protein.

### BRIEF DESCRIPTION OF THE FIGURE

FIG. 1: Kinetics of ethanol production of strains 1b, 18b, 102, 15, 14b. The error bar represents a standard deviation of three independent cultures.

### DETAILED DESCRIPTION

While working on the expression of certain bacterial chaperone proteins in a eukaryotic cell, the Inventors discovered that, in a quite astonishing manner, bacterial chaperones can be expressed in the cytosol of a eukaryotic cell and retain their chaperone function in said eukaryotic cell. The bacterial chaperone function is in addition to the cytosolic chaperone function already present in the eukaryotic cell. The Inventors further discovered that the expression of a particular triplet of chaperones, namely the bacterial chaperones GroEL and GroES and the chaperone RbcX, confers upon the transformed eukaryotic cell which expresses them particularly advantageous properties in terms of growth, expression and functional protein folding.

The effects observed obligatorily require the simultaneous presence of the three chaperones cited, coming preferentially from at least two different microorganisms. If it was known that co-expression of chaperones is likely to improve in a bacterial system the folding of heterologous proteins and thus potentially the performance of a bioprocess dependent thereon, it was not foreseeable that a specific inter-organism combination of three bacterial chaperone proteins would prove particularly effective by being expressed in a eukaryotic context. The molecular mechanisms resulting in the effects of the invention are currently unknown even if they are probably, at least in part, related to protein folding or to the control of their intracellular destiny. A surprising general role is played by the protein of the RbcX family (described in the prior art as specialized for the functional association of the RuBisCO complex of photosynthetic organisms), in the presence of the chaperones GroES and GroEL which play complementary roles. This feature of the invention suggests that the observed effect cannot be wholly attributed to a role of facilitated folding and could involve other mechanisms like a modulation of the lifespan of specific proteins, the assembly of complexes or the modification of the properties thereof, or specific effects of a nature to be defined.

In this context, the invention thus proposes to transform eukaryotic cells so that they express a particular triplet of chaperones, namely the bacterial chaperones GroEL and GroES and the chaperone RbcX. Such transformed cells can find many applications, in particular in the context of the production of recombinant proteins.

The invention thus has as an object a eukaryotic cell, characterized in that it expresses the chaperones RbcX, GroES and GroEL.

The chaperones GroEL and GroES belong to the family of heat-shock proteins (HSP). These chaperones are present in many bacteria. In the context of the invention, these chaperones are referred to as “general chaperones” in the sense that they are known to co-act in order to enable the effective folding of a very large number of proteins (M. Mayhew et al. 1996, “Protein folding in the central cavity of the GroEL-GroES chaperonin complex” Nature 1996 Feb. 1; 379(6564):420-6). According to the invention, the chaperones GroEL and GroES can come from any bacterium expressing them, and in particular for example, from E. coli (Gene ID: 948655 and 948665), S. elongatus (Gene ID: 3199735, 3199535 and 3198035), S. pneumonia (GenBank accession number: AF117741), S. pyogenes (GenBank accession number: SPGROELGN), S. aureus (GenBank accession number: STAHSP) or P. aeruginosa (GenBank accession number: ATCC9027). The person skilled in the art is perfectly capable of identifying in a bacterium the nucleic sequences likely to encode one or another of these chaperones. For informational purposes, it will be noted that the sequence similarity of the chaperones (% of amino acids identical in the alignment) is 61% between GroEL1 from S. elongatus and GroEL from E. coli; 56% between GroEL2 from S. elongatus and GroEL from E. coli; 63% between GroEL1 and GroEL2 from S. elongatus.

The cells according to the invention also express the chaperone RbcX known in cyanobacteria and plants to participate in the correct assembly of the RbcL and RbcS subunits of Rubisco. In the context of the invention, this chaperone is referred to as a “specific chaperone” in the sense that this protein is known to play a role in the functional association of protein complexes, as is notably the case with Rubisco (S. Saschenbrecker et al. 2007, “Structure and function of RbcX, an assembly chaperone for hexadecameric Rubisco”, Cell. 2007 Jun. 15; 129(6):1189-200). According to the invention, the chaperone RbcX can come from any cyanobacterium expressing it, and in particular, for example, from S. elongatus (SEQ ID NO: 3), Synechocystis sp. (Kaneko et al., “Sequence analysis of the genome of the unicellular cyanobacterium Synechocystis sp. strain PCC6803. II. Sequence determination of the entire genome and assignment of potential protein-coding regions.” DNA Res. 3(3), 109-136 (1996)), Anabaena sp. (Li et al. J. Bacteriol. (1997), 179(11), 3793-3796), Microcystis sp., Tychonema sp., Planktothrix sp. or Nostoc sp. (Rudi et al., J. Bacteriol. (1998), 180(13), 3453-3461).

In the context of the invention, “chaperone activity” refers to action on protein folding and/or on the functional association of protein complexes.

In a particular embodiment of the invention, the chaperones used come preferentially from two different organisms. According to the invention, the chaperones can come from one or more different bacteria. Preferably, the three chaperones come from at least two distant Gram-negative bacterial species, of which at least one is a cyanobacterium.

According to another particular implementation of the present invention, at least one of the general chaperones GroES and GroEL comes neither from a cyanobacterium nor from another bacterium expressing a RuBisCO complex. According to the invention, the chaperones GroES and GroEL can come from the same bacterium or from two different bacteria. In a particular exemplary embodiment, the chaperones GroES and GroEL come from E. coli.

In a particular exemplary embodiment, the chaperones GroES and GroEL come from E. coli and the chaperone RbcX comes from Synechococcus elongatus.

In another exemplary embodiment, the three chaperones GroES, GroEL and RbcX come from Synechococcus elongatus. In such a case, the transformed cell can express one or the other or both isoforms (GroEL1 and GroEL2) of the chaperone GroEL, preferentially both isoforms.

In the context of the invention, a protein is considered to “come” from a given organism when it has an amino acid sequence identity greater than 95% and the same function as the protein considered from said organism.

In the present text, “GroES” and “GroEL” refer to any protein having chaperone activity and having between 65% and 100% amino acid identity with GroES and GroEL from E. coli K 12, respectively. Alternatively, “GroES” and “GroEL” refer to general chaperones having a lower percent identity, and in particular between 55% and 65%, and more particularly between 56% and 63%, such as the general chaperones from S. elongatus. The chaperone activity of a variant of the general chaperones GroES and GroEL from E. coli could be confirmed, for example, by substituting in the various examples described below the expression cassette encoding native GroES or GroEL from E. coli with variants of chaperones to be evaluated.

The chaperone RbcX is very distant from GroEL and GroES and its sequence cannot be aligned with the sequences of these two chaperones.

In the present text, “RbcX” refers to any protein, in particular from a cyanobacterium, having chaperone activity and having more than 50% amino acid sequence identity with the chaperone RbcX encoded by the sequence SEQ ID NO: 3 and retaining the specific chaperone activity of this protein. The specific chaperone activity can be confirmed in a yeast expressing the RbcL and RbcS subunits of RuBisCO from S. elongatus, by replacing the expression cassette including the sequence SEQ ID NO: 3 with any other sequence to be evaluated, and by measuring with an in vitro test on cellular extracts the RuBisCO activity thus obtained.

Preferably, the present invention is implemented with a chaperone RbcX the amino acid sequence identity with the chaperone RbcX encoded by SEQ ID NO: 3 of which is greater than 80%, preferentially greater than 90%, more preferentially greater than 95%, even more preferentially greater than 99%.

The invention can be implemented with any type of eukaryotic cell from a unicellular or multicellular organism. In particular, the combination of chaperones according to the invention can be expressed in a yeast cell, a fungal cell, a plant cell, an animal cell such as a mammalian cell, etc.

In particular, the present invention relates to a transformed yeast expressing a specific chaperone RbcX, a bacterial general chaperone GroES, and a bacterial general chaperone GroEL.

More particularly, the present invention relates to a transformed yeast, characterized in that it contains:

(i) an expression cassette containing a sequence encoding the chaperone RbcX involved in the folding of a bacterial form I RuBisCO enzyme, under the transcriptional control of a suitable promoter;
(ii) an expression cassette containing a sequence encoding a bacterial general chaperone GroES, under the transcriptional control of a suitable promoter; and
(iii) an expression cassette containing a sequence encoding a bacterial general chaperone GroEL, under the transcriptional control of a suitable promoter.

The invention can be implemented with any yeast of interest. Advantageously, the yeast is selected from Saccharomyces, Yarrowia and Pichia. For example, the transformed yeast according to the invention is a Saccharomyces cerevisiae cell. In another example, the transformed yeast according to the invention is a Yarrowia lipolytica cell, or a Pichia pastoris cell.

The use of Pichia pastoris is particularly advantageous for the production of recombinant proteins. Indeed, Pichia has a high-performance eukaryotic protein expression system in terms of both secretion and intracellular expression. It is particularly suitable for large-scale production of recombinant eukaryotic proteins. In particular, Pichia can be used for the production of excreted proteins with high yields, in order to reduce production costs and times compared to those associated with mammalian cell expression systems.

Yarrowia lipolytica is also suitable for use for the production of recombinant proteins. Indeed, Yarrowia has (i) high-density growth, (ii) a high secretion rate, (iii) an absence of the alkaline protease AEP and (iv) an ability to produce S. cerevisiae invertase which allows the use of sucrose as a carbon source (Nicaud et al., 1989). The latter property is particularly advantageous in the case of industrial production, because this strain can grow efficiently on inexpensive substrates such as molasses.

The invention also relates to a eukaryotic cell from a multicellular organism, such as an animal cell and in particular a mammalian cell, transformed, expressing a specific chaperone RbcX, a bacterial general chaperone GroES and a bacterial general chaperone GroEL.

In an exemplary embodiment, such a eukaryotic cell is transformed to contain:

(i) an expression cassette containing a sequence encoding the chaperone RbcX involved in the folding of a bacterial form I RuBisCO enzyme, under the transcriptional control of a suitable promoter;
(ii) an expression cassette containing a sequence encoding a bacterial general chaperone GroES, under the transcriptional control of a suitable promoter; and
(iii) an expression cassette containing a sequence encoding a bacterial general chaperone GroEL, under the transcriptional control of a suitable promoter.

The invention relates in particular to a transformed CHO cell expressing the triplet of chaperones according to the invention.

According to the invention, genes encoding the chaperones GroEL, GroES and RbcX are introduced into the eukaryotic cells in a form enabling their expression in said cells. Thus, the sequences encoding the chaperones are associated with promoter sequences enabling their transcription. In an embodiment, the same promoter sequence is associated with the sequences encoding the three chaperones. In another embodiment, the chaperone RbcX is associated with a particular promoter different from the promoter(s) associated with the chaperones GroEL and GroES. In another embodiment, each chaperone is associated with a different particular promoter.

Promoters usable in the context of the present invention include constitutive promoters, namely promoters which are active in most cellular states and environmental conditions, as well as inducible promoters which are activated or repressed by exogenous physical or chemical stimuli, and which thus induce a variable level of expression as a function of the presence or absence of these stimuli.

For expression cassettes in yeasts, examples of constitutive promoters are those of the genes TEF1, TDH3, PGI1, PGK, ADH1. Examples of inducible promoters are the promoters tetO-2, GAL10, GAL10-CYC1, PHO5. Preferably, the promoters used will be different from one cassette to another. The expression cassettes of the invention further comprise common sequences such as transcription terminators, and if need be other transcription regulatory elements. The expression cassettes in accordance with the invention can be inserted into chromosomal DNA of the host cell, and/or carried by one or more extrachromosomal replicon(s). The relative stoichiometry of these proteins is likely to play an important role in the optimal implementation of the present invention. The co-expression systems described in the experimental section below are particularly relevant in this respect. The invention however is not limited to the use of these systems, and it can be implemented with any variant of expression of the elements mentioned having effects at least equivalent, such that they can be measured, for example, by measuring the growth of a transformed cell in standard medium for said cell.

According to an advantageous embodiment, the three expression cassettes form a continuous block of genetic information. It can also be advantageous that the expression cassettes of the three chaperones are carried by a single episomal genetic element. A particularly advantageous aspect of the present invention is thus a single “genetic plug-in” (continuous DNA sequence) carried by an episomal element having a centromeric origin of replication. Transformation by this element is sufficient to introduce the properties of interest in wild yeasts or those carrying any engineering.

According to the invention, the genes encoding each of the chaperones can be introduced in one or more copies into the cell. In particular, it is possible to introduce one, two, three or more cassettes containing a sequence encoding GroES, one, two, three or more cassettes containing a sequence encoding GroEL, and one, two, three or more cassettes containing a sequence encoding RbcX. Likewise, a cassette can contain several copies of a sequence encoding GroES, GroEL or RbcX. In the case where several copies of genes encoding a chaperone are introduced into the cell, the same sequence, i.e. coming from the same bacterium, is preferentially used each time. It is also possible to use sequences coming from different bacteria.

As mentioned above, cells according to the present invention have improved properties (growth rate, resistance, production capacity, etc.). These cells are thus particularly useful for producing proteins or other compounds, or for improving fermentation processes.

### Production of Proteins

The invention also relates to a eukaryotic cell transformed to express a combination of chaperones as described above and which further comprises at least one expression cassette for a heterologous protein other than said chaperones, and/or which has undergone a sequence engineering modifying the level of expression and/or the sequence of an endogenous protein.

According to a preferred implementation of the invention, the transformed eukaryotic cell is a yeast. According to another preferred implementation of the invention, the transformed eukaryotic cell is a CHO cell.

Thus, the invention also has as an object a yeast or a CHO cell transformed to express a combination of chaperones as described above, and containing:

(iv) an expression cassette for a heterologous protein other than said chaperones; and/or
(v) having undergone a sequence engineering modifying the level of expression and/or the sequence of an endogenous protein.

According to the invention, it is possible to use such a transformed cell to produce one or more proteins of interest.

Advantageously, the protein of interest is not Rubisco. Likewise, preferentially, the protein of interest is a protein other than PKR. Also, if the transformed eukaryotic cell expresses Rubisco and/or PKR, it advantageously expresses at least one other heterologous protein.

In a particular embodiment, the transformed cell expressing the triplet of chaperones GroES, GroEL and RbcX is further modified so as to express and excrete a recombinant protein.

The present invention also relates to a biotechnological process for producing at least one compound selected from chemical molecules, enzymes, hormones, antibodies and proteins, characterized in that it comprises a step of culturing a transformed cell as described above, under conditions enabling the synthesis, by said cell, of this compound, and optionally a step of collecting and/or purifying said compound.

The invention also relates to a process for producing a recombinant protein comprising (i) inserting a sequence encoding said protein into a eukaryotic cell expressing the triplet of chaperones RbcX, GroES and GroEL, (ii) culturing said cell under conditions enabling the expression of said sequence and optionally (iii) collecting and/or purifying said protein. Advantageously, said protein is an enzyme or a hormone.

In a particular embodiment, the cell according to the invention is transformed so as to produce at least one heterologous enzyme. For example, the cell is transformed to produce an enzyme selected from endotoxins, such as Bacillus thuringiensis endotoxin, lipases, subtilisins, cellulases and luciferases.

In another particular embodiment, the cell according to the invention is transformed so as to produce at least one molecule of medical interest. For example, the cell is transformed to produce a hormone, a growth factor, an antibody, etc. Preferentially, the molecule of medical interest is selected from erythropoietin, type I and/or II alpha-interferons, granulocyte colony-stimulating factors, insulin, growth hormones, tissue plasminogen activators.

Advantageously, the transformed cell according to the invention has improved production of the protein(s) of interest, compared to a recombinant cell not expressing the combination of chaperones of the invention. In the context of the invention, “improved” production means in terms of quantity and/or quality. In particular, the transformed cell according to the invention can produce proteins of interest which are more active and/or more stable, and thus less likely to be degraded, which enables a greater accumulation of said proteins, compared to the heterologous proteins produced by a recombinant cell not expressing the combination of chaperones of the invention. Thus, the increase in the level of expression of a recombinant protein by a transformed eukaryotic cell according to the invention is explained in particular by a greater stability and thus a greater accumulation of said proteins in the cell and/or the culture medium. Moreover, the expression of the combination of chaperones by the transformed cell can advantageously increase the resistance of the recombinant cell to the recombinant proteins that it expresses, thus also participating in increasing production yield.

The present invention also relates to the use of a combination of expression cassettes enabling the expression, in a eukaryotic cell, of the specific chaperone RbcX and the general chaperones GroES and GroEL, to improve the physiology and/or the performance (in particular the growth rate) of said eukaryotic cell. According to a preferred implementation of this aspect of the invention, the eukaryotic cell the improved physiology of which is sought is a yeast. According to another preferred implementation, said eukaryotic cell has not been transformed to express a sequence encoding the RbcL subunit of a bacterial form I RuBisCO enzyme, and/or a sequence encoding the RbcS subunit of said RuBisCO enzyme.

As mentioned above, a combination of expression cassettes enabling the expression of the general chaperones GroES and GroEL and the specific chaperone RbcX is particularly useful for improving the physiology of a eukaryotic cell having undergone a sequence engineering modifying the level of expression and/or the sequence of an endogenous protein or comprising at least one expression cassette for a heterologous protein other than said chaperones, for example in the form of an episomal genetic element.

In particular, the concomitant expression, in a cell, of the general chaperones GroES and GroEL and the specific chaperone RbcX makes it possible to increase the growth rate of said cell and/or to increase the resistance of said cell to an environmental stress, in particular to a stress due to the toxicity of an element present in the culture medium of the cell. Another advantageous application is to increase the resistance of the cell to the toxicity of a compound synthesized thereby, and thus the production of a compound of interest.

A great many applications of the invention can be envisaged, in particular all the applications related to improving the folding or the stability (resistance to chemical or thermal agents, intrinsic lifespan) of proteins homologous or heterologous to the transformed eukaryotic cell. They can be proteins themselves, either of interest in enzymatic catalysis, or because of their intrinsic properties (antibodies, therapeutic proteins, structural proteins within complexes, etc.); Applications related to the improved performance of a synthetic or semi-synthetic metabolic chain (involving proteins of the transformed eukaryotic cell); in this case, the advantage is chiefly an improvement in the result of their actions on the production of a product of interest (chemical molecule). This effect can result from the improvement in folding or stability but also from other phenomena, for example subcellular transport, facilitated or modified formation of complexes, modulation of coupling mechanisms, etc.; Applications resulting from positive overall effects on an organism in terms of growth, viability, adaptability, resistance to or recovery from stress, formation of products of interest without the mechanism being known or explainable.

Examples of industrial applications of the invention include, but are not limited to:

### Effect on Protein Folding/Stability

The production of certain recombinant proteins of interest considered as difficult or even impossible to produce in soluble and functional form can be improved by the implementation of the invention. This is explained in particular by a correction of the folding defects of said proteins in the transformed eukaryotic cell according to the invention. This can be particularly useful in the fields of health, energy, chemicals, food processing, etc.

Protection Against Toxicity Induced by Products of an Engineering of Interest or Intermediates of this Engineering

It may be a question of the bioproduction of molecules toxic to the host cell or whose production process involves toxic intermediates, for example medicines (hydrocortisone, artemisinic acid or trictosinide, certain flavonoids to take developed processes) or reactive molecules such as unsaturated ketones, aldehydes (for example vanillin), etc. Other examples correspond to processes producing molecules that become toxic at high concentration, for example ethanol or other alcohols. Ethanol production, for example, is limited by the tolerance of yeast strains to high alcohol concentrations. Other examples relate to the production of highly varied industrial chemical molecules, precursors of current products or chemical intermediates and of course biofuels. They can also be recombinant proteins the production of which by a recombinant cell tends to unbalance the metabolism, inducing cell death. The use of a eukaryotic cell expressing the combination of chaperones according to the invention advantageously makes it possible to increase the resistance of the transformed cell to toxicity induced by the recombinant proteins which it expresses.

### Heat Tolerance

Improving the heat tolerance of specific enzymes or of organisms such as yeasts is an important factor for the biotechnological productivity of many poorly soluble molecules, for example.

Increasing the Biomass of a Microorganism Produced from a Fixed Quantity of Carbon Source

These are effects which are not understood at the molecular level but which are observed in the first experiments recounted below.

As mentioned above, the present invention also relates to a nucleic acid molecule comprising:

(i) an expression cassette containing a sequence encoding the chaperone RbcX involved in the folding of a bacterial form I RuBisCO enzyme, under the transcriptional control of a suitable promoter;
(ii) an expression cassette containing a sequence encoding a bacterial general chaperone GroES, under the transcriptional control of a suitable promoter; and
(iii) an expression cassette containing a sequence encoding a bacterial general chaperone GroEL, under the transcriptional control of a suitable promoter.

An example of such a “genetic plug-in” is a continuous DNA sequence comprising the three cassettes mentioned above (in any order), carried by an episomal element having a centromeric origin of replication.

The experimental section below illustrates but does not limit the invention, by presenting:
The constructions created and the methods implemented (Example 1);
A first series of tests comparing the effect of various combinations of chaperones on reconstruction of the activity of a type I RuBisCO consisting of the assembly of 16 peptide chains belonging to two different types. The expression of various associations of chaperones was combined with the expression of the RbcL and RbcS polypeptides of a “type I RuBisCO”. This made it possible to analyze the role of the combination of chaperones on the RuBisCO activity measured in vitro on yeast cell extracts (Example 2);
A second series of tests involving the co-expression of the chaperone systems in a yeast conditionally expressing a phosphoribulokinase (PRK) of S. elongatus. In the absence of the invention, induction of PRK expression induces in the yeast a major toxic effect ranging from very slow growth to total lethality (>99.99%) depending on the S. cerevisiae strains and the culture conditions (medium, oxygen level, pH). The exemplification illustrates that the invention “cures” this morbid state without interfering with the activity of the PRK enzyme, which remains fully active (Example 3);
A third series of tests showing that the invention restores a wild level of growth in a strain carrying autotrophies complemented by even neutral plasmids (themselves not leading to the expression of functions other than their own replication). This situation is typical of metabolic engineering involving episomal genetic elements. In the example, the invention totally corrects the negative impact on growth related to the presence of episomal genetic structures (Example 4); and
Another series of tests illustrating that the cells of the invention are particularly useful for the production of compounds and in particular recombinant proteins (Example 5).

### EXAMPLES

In the examples below, and unless otherwise specified, the same acronyms are used from one table to another and from one experiment to another to designate the same elements.

### Example 1: Materials and Methods—Construction of the “CHAPERONES Plug-In” and Vectors—Constructions of the Various Strains—Culture and Measurement Methods

1.1. Construction of the “CHAPERONES Plug-In” and Vectors for S. cerevisiae

Certain constructions described below enable the expression of the two RbcS and RbcL subunits of RuBisCO (pFPP45) and that of phosphoribulokinase (PRK) (pFPP20) from Synechococcus elongatus pCC6301. Other constructions described below were created in order to make it possible to work out, from a single expression vector, variable combinations of expression of the specific chaperone RbcX from Synechococcus elongatus and the general chaperones from E. coli GroES (Gene ID: 948655), GroEL (Gene ID: 948665) or their homologues GroES (Gene ID: 3199735), GroEL1 (Gene ID: 3199535) and GroEL2 (Gene ID: 3198035) from Synechococcus elongatus.

Synthetic genes encoding the RbcS (Gene ID: 3200023) and RbcL (Gene ID: ID: 3200134) subunits and the specific chaperone RbcX (Gene ID: 3199060) of RuBisCO from Synechococcus elongatus pCC6301, and optimized for expression in yeast, were prepared and cloned into the plasmid pBSII (Genecust). Variants optimized for expression in yeast, in which an HA tag was added at 3′ end of the coding sequence, were also constructed. The sequences of these synthetic genes (without the HA tag) are respectively indicated in the sequence listing in the Appendix under numbers SEQ ID NO: 1 to SEQ ID NO: 3.

Likewise, synthetic genes encoding phosphoribulokinase (PRK) (SEQ ID NO: 4) (pFPP20), as well as the general chaperones GroES (SEQ ID NO: 5), GroEL1 (SEQ ID NO: 6) and GroEL2 (SEQ ID NO: 7) from Synechococcus elongatus pCC6301, and optimized for expression in yeast, were constructed and cloned.

The sequences encoding the chaperones GroES and GroEL from E. coli were amplified from E. coli cultures and cloned into the plasmid pSC-B-amp/kan (Stratagene).

The sequences recovered from the cloning vectors were introduced into yeast expression vectors. These host vectors are listed in Table I below.

TABLE I
List of vectors used

| | Yeast | | | |
|---|---|---|---|---|
| | replication | Selection | Transcription cassette | E. coli |
| Names | origin | marker | (promotor-terminator) | replicon |
| pFPP5 | 2 μ | URA3 | pGAL10-CYC1-tPGK | Yes (AmpR) |
| pFPP10 | 2 μ | URA3 | pTDH3--tADH | Yes (AmpR) |
| pFPP11 | 2 μ | URA3 | pTDH3--tCYC1 | Yes (AmpR) |
| pFPP12 | 2 μ | URA3 | pTGI1--tCYC1 | Yes (AmpR) |
| pFPP13 | ARS-CEN6 | LEU2 | pTEF1-tPGK | Yes (AmpR) |

Note:
pGAL10-CYC1: synthetic promoter composed of the UAS of the GAL10 gene and the transcription initiation of the CYC1 gene (Pompon et al., Methods Enzymol, 272, 51-64, 1996).

The expression cassettes thus obtained are listed in Table II below.

TABLE II
Expression cassettes

| Names | Promoter | Open reading frame | Tag | Terminator |
|---|---|---|---|---|
| CAS6 | TDH3p | RbcL | None | ADH1 |
| CAS7 | TetO7p | PRK | None | CYC1t |
| CAS16 | TEF1 | RbcS | None | PGK |
| CAS19 | TEF1p | RbcX | None | PGK |
| CAS21 | PGI1p | GroES E. coli | None | CYC1 |
| CAS22 | TDH3 | GroEL E. coli | None | ADH |
| CAS23 | PGI1p | GroES S. elongatus | None | CYC1t |
| CAS25 | TDH3p | GroEL2 S. elongatus | None | ADH1t |
| CAS28 | PGI1p | polylinker | None | CYC1t |
| CAS33 | TEF1p | polylinker | None | PGKt |

In certain vectors, two or three cassettes were inserted. To that end, the plasmids were amplified in the bacterium Escherichia coli DH5a and prepared by maxiprep, then digested by suitable restriction enzymes. Lastly, the fragments are integrated into host vectors by ligation by T4 ligase (FERMENTAS) or by homologous recombination directly in yeast. The list of vectors constructed is indicated in Table III below.

TABLE III
Expression vectors

| Names | Origin type | Cassette 1 | Cassette 2 | Cassette 3 | Markers | Host vector |
|---|---|---|---|---|---|---|
| pFPP13 | ARS415-CEN6 | CAS33 | None | None | LEU2 | pFL36 |
| pFPP20 | ARS416-CEN4 | CAS7 | None | None | TRP | pCM185 |
| pFPP45 | 2μ | CAS6 | CAS16 | None | URA3 | pFPP5/pFPP10 |
| pFFP53 | ARS415-CEN6 | CAS19 | CAS28 | None | LEU2 | pFL36 |
| pFPP56 | ARS415-CEN6 | CAS19 | CAS21 | CAS22* | LEU2 | pFPP13 |
| pFB05 | ARS415-CEN6 | CAS19 | CAS25* | CAS21 | LEU2 | pFFP56 |
| pFB07 | ARS415-CEN6 | CAS23 | CAS22* | CAS19 | LEU2 | pFFP56 |
| pFB08 | ARS415-CEN6 | CAS23 | CAS25* | CAS19 | LEU2 | pFFP56 |
| pFB09 | ARS415-CEN6 | CAS21 | CAS22* | None | LEU2 | pFFP56 |

1.2. Construction of Various S. cerevisiae Strains

The various plasmids above were constructed so as to enable the on-demand expression of individual components or an association of these components within yeast strains. Various vectors or combinations of vectors were thus used to transform several strains of the yeast S. cerevisiae (W303.1B, FY1679 and CEN.PK 1605). CEN.PK 1605 is the prototrophic version of strain 1605. It is thus a positive control for “physiological behavior”. Each number, compiled in the first column of Table IV below, corresponds to the association of vectors described on the corresponding line of the same table. For each strain used, therefore, the associated reference number provides information about the reconstructed engineering. Only the CEN.PK 1605 strains have been exemplified (Table IV) in the interest of clarity, but the nomenclature is the same for the other two strains.

TABLE IV
Combination of plasmids and strains (references to Table III.)

| | | | | | Proteins expressed | | | | | |
|---|---|---|---|---|---|---|---|---|---|---|
| Combination | Parental | Vector | Vector | Vector | Rbc | Rbc | Rbc | PRK | GroE | GroE |
| number | strain | 1 | 2 | 3 | S | L | X | syn | S | L |
| 1b | CEN. PK | PYEDP51 | pCM185 | pFPP13 | | | | | | |
| | 1605 | | | | | | | | | |
| 2 | CEN. PK | pFPP45 | pCM185 | pFPP56 | X | X | X | | E. | E. |
| | 1605 | | | | | | | | coli | coli |
| 3 | CEN. PK | pFPP45 | pFPP20 | pFPP56 | X | X | X | syn | E. | E. |
| | 1605 | | | | | | | | coli | coli |
| 4 | CEN. PK | pFPP45 | pFPP20 | pFPP53 | X | X | X | syn | | |
| | 1605 | | | | | | | | | |
| 5 | CEN. PK | pFPP45 | pCM185 | pFPP53 | X | X | X | | | |
| | 1605 | | | | | | | | | |
| 12 | CEN. PK | PYEDP51 | pCM185 | pFPP53 | | | X | | | |
| | 1605 | | | | | | | | | |
| 13b | CEN. PK | PYEDP51 | pCM185 | pFPP56 | | | X | | E. | E. |
| | 1605 | | | | | | | | coli | coli |
| 14b | CEN. PK | PYEDP51 | pFPP20 | pFPP53 | | | X | Syn | | |
| | 1605 | | | | | | | | | |
| 15 | CEN. PK | PYEDP51 | pFPP20 | pFPP56 | | | X | syn | E. | E. |
| | 1605 | | | | | | | | coli | coli |
| 16b | CEN. PK | pFPP45 | pCM185 | pFPP13 | X | X | | | | |
| | 1605 | | | | | | | | | |
| 17b | CEN. PK | pFPP45 | pFPP20 | pFPP13 | X | X | | syn | | |
| | 1605 | | | | | | | | | |
| 18b | CEN. PK | PYEDP51 | pFPP20 | pFPP13 | | | | syn | | |
| | 1605 | | | | | | | | | |
| 101 | CEN. PK | pFPP45 | pFPP20 | pFB08 | X | X | X | Syn | syn | L2 |
| | 1605 | | | | | | | | | syn |
| 102 | CEN. PK | PYEDP51 | pFPP20 | pFB09 | | | | Syn | E. | E. |
| | 1605 | | | | | | | | coli | coli |
| 102b | CEN. PK | pFPP45 | pFPP20 | pFB09 | X | X | | syn | E. | E. |
| | 1605 | | | | | | | | coli | coli |
| 103 | CEN. PK | PYEDP51 | pCM185 | pFB09 | | | | | E. | E. |
| | 1605 | | | | | | | | coli | coli |

(Syn: Synechococcus elongatus;
L2 syn: GroEL2 Synechococcus elongatus,
L1 syn: GroEL1 Synechococcus elongatus)

Notes relating to certain tables:

1. pCM185: Commercial plasmid (ATCC 87659)
2. pFL36: Commercial plasmid (ATCC 77202)
3. PYeDP51: “Empty” plasmid, described in the following article: Urban P, Mignotte C, Kazmaier M, Delorme F, Pompon D. Cloning, yeast expression, and characterization of the coupling of two distantly related Arabidopsis thaliana NADPH-cytochrome P450 reductases with P450 CYP73A5. J Biol Chem. 1997 Aug. 1; 272(31):19176-86.
4. GroES E. coli Gene ID: 6061370; GroEL E. coli Gene ID: 6061450
5. S. Cerevisiae strain CEN.PK 113-7D: Mat a prototrophic
6. S. Cerevisiae strain CEN.PK 1605: Mat a HIS3leu2-3.112trp1-289 ura3-52 MAL.28c. Strain resulting from CEN.PK 113-7D
7. The other abbreviations refer to S. cerevisiae genes described in the data banks.
8. Synthetic genes: The Synechococcus elongatus genes encoding the RuBisCO subunits, the chaperone specific to RuBisCO assembly (RbcX), the PRK and the general chaperones GroES, GroEL1 and GroEL2 were synthesized after proprietary re-encoding for yeast implementing an inhomogeneous codon bias and cloned into pCC6301 (commercial). The coding sequences of these proteins (after re-encoding) are presented in the Appendix (SEQ ID NO: 1: RbcS coding sequence; SEQ ID NO: 2: RbcL coding sequence; SEQ ID NO: 3: RbcX coding sequence; SEQ ID NO: 4: PRK coding sequence; SEQ ID NO: 5: GroES coding sequence; SEQ ID NO: 6: GroEL1 coding sequence; SEQ ID NO: 7: GroEL2 coding sequence).
9. The coding sequences of E. coli chaperones GroES and GroEL were amplified from the bacterium, cloned in pSC-B-amp/kan (Stratagene) and assembled without re-encoding in the expression vectors (see above).
10. The re-encoded sequences of cDNAs encoding Synechococcus elongatus chaperonins were inserted by homologous recombination into previously linearized vector pUC57 by co-transforming the two molecules in yeast. Similarly, the ORFs were amplified by PCR from previous constructions, generating flanking regions homologous to the promoters and terminators carried by vector pFPP56. That allowed cloning by homologous recombination by co-transforming this PCR product in a yeast strain with previously linearized vector pFPP56, generating the various expression vectors described in Table III according to the cassettes described in Table II.
1.3. Construction of Various Saccharomyces cerevisiae Strains Integrating Chaperones of Different Origins

Chaperones from various bacteria were evaluated. Thus, combinations including proteins from the same species (S. elongatus) were also tested, by combining RbcX from S. elongatus with GroES and GroEL1 and/or GroEL2 from S. elongatus.

TABLE V
Expression cassettes

| Names | Promoter | Open reading frame | Tag | Terminator |
|---|---|---|---|---|
| CAS19 | TEF1p | RbcX S. elongatus optimized | None | PGKt |
| CAS21 | PGI1p | GroES E. coli | None | CYC1t |
| CAS22 | TDH3p | GroEL E. coli | None | ADH1t |
| CAS23 | PGI1p | GroES S. elongatus optimized | None | CYC1t |
| CAS24 | TEF2p | GroEL1 S. elongatus optimized | None | TEF1t |
| CAS25 | TDH3p | GroEL2 S. elongatus optimized | None | ADH1t |

TABLE VI
Expression vectors

| | Origin | | | | Cassette | Auxotrophy | Host |
|---|---|---|---|---|---|---|---|
| Names | type | Cassette 1 | Cassette 2 | Cassette 3 | 4 | markers | vector |
| pFFP56 | ARS415- | CAS19 | CAS21 | CAS22* | | LEU2 | pFL36 |
| | CEN6 | | | | | | |
| pFB08 | ARS415- | CAS23 | CAS25* | CAS19 | | LEU2 | pFFP56 |
| | CEN6 | | | | | | |
| pCB02 | ARS415- | CAS23 | CAS25* | CAS19 | CAS24 | LEU2 | pFB08 |
| | CEN6 | | | | | | |

TABLE VII
Combination of plasmids and strains

| Combi- | Parental | Vector | Vector | Vector | Proteins expressed | | |
|---|---|---|---|---|---|---|---|
| nation | strain | 1 | 2 | 3 | RbcX | GroES | GroEL |
| 1b | CEN. PK | V51TEF | pCM185 | pFL36 | | | |
| | 1605 | | | | | | |
| 13b | CEN. PK | V51TEF | pCM185 | pFPP56 | syn | E. coli | E. coli |
| | 1605 | | | | | | |
| 116 | CEN. PK | V51TE | pCM185 | pFB08 | Syn | syn | L2 syn |
| | 1605 | | | | | | |
| 111 | CEN. PK | V51TEF | pCM185 | pCB02 | Syn | syn | L2 syn |
| | 1605 | | | | | | L1 syn |

(Syn: Synechococcus elongatus;
L2 syn: GroEL2;
L1 syn: GroEL1 Synechococcus elongatus)

1.4. Constructions of Pichia pastoris Strains

In order to maintain functional expression of the genes contained in the plug-in, the promoters and terminators were replaced with promoters and terminators functional in Pichia pastoris GS115 (Thermo Fisher Scientific C181-00).

On plasmid pFPP56 (Table III), each promoter controlling expression of the RbcX, GroES and GroEL genes, from CAS19, CAS20 and CAS21 (Table II), was replaced with a compatible promoter according to the literature (Table VIII below).

TABLE VIII
Expression cassettes

| Names | Promoters | Open reading frame | Terminator |
|---|---|---|---|
| CAS50 | PEX8 | RbcX | TEF1 |
| CAS51 | AOX1 | GroES | PGK |
| CAS52 | FLD1 | GroEL | ADH1 |

The region including the three expression cassettes below (Table IX) was amplified by PCR and NotI cloned in commercial plasmid pPIC3.5 (Thermo Fisher K1710-01).

TABLE IX
Expression vectors

| | | Cassette | Cassette | Cassette | | Host |
|---|---|---|---|---|---|---|
| Names | Origin type | 1 | 2 | 3 | Marker | vector |
| pCB05 | ARS415- | CAS50 | CAS51 | CAS52 | LEU2 | pFL36 |
| | CEN6 | | | | | |
| pCB06 | Integrative | CAS50 | CAS51 | CAS52 | HIS4 | pPIC3.5 |

These plasmids were previously linearized and transformed individually in Pichia pastoris strain GS115 (Thermo Fisher C181-00) auxotrophic for histidine according to the EasySelect Pichia Expression Kit protocol (Thermo Fisher) and selected on minimal medium and glucose at 30° C.

TABLE X
Plasmids and strains

| | | | Proteins expressed | | |
|---|---|---|---|---|---|
| Names | Parental strains | Vector 1 | RbcX | GroES | GroEL |
| PPGC115_01 | Pichia pastoris | pIC3.5 | — | — | — |
| | GS115 | | | | |
| PPGC115_02 | Pichia pastoris | pCB06 | S. elongatus | E. coli | E. coli |
| | GS115 | | | | |

1.5. Construction of Yarrowia lipolytica Strains

Wild strain W29 (ATCC 20460, MatA) isolated from wastewater was used.

On plasmid pFPP56 (Table III), each promoter controlling the expression of the RbcX, GroES and GroEL genes, from CAS19, CAS20 and CAS21 (Table II), was replaced with a compatible promoter according to the literature (Table XI below).

TABLE XI
Expression cassettes

| Names | Promoters | Open reading frame | Terminator |
|---|---|---|---|
| CAS55 | TEF | RbcX | TEF1 |
| CAS56 | EXP | GroES | PGK |
| CAS57 | GDP | GroEL | ADH1 |

The region including the three expression cassettes above (Table XI) was amplified by PCR and cloned in commercial plasmid pYLEX1.

TABLE XII
Expression vector

| | Origin | Cassette | Cassette | Cassette | | Host |
|---|---|---|---|---|---|---|
| Names | type | 1 | 2 | 3 | Marker | vector |
| pCB07 | Integrative | CAS19 | CAS20 | CAS21 | LEU2 | pYLEX1 |

These plasmids were linearized and transformed individually in a Yarrowia lipolytica strain auxotrophic for leucine according to the YLOS Transformation Kit protocol (Yeastearn Biotech) and selected on minimal medium and glucose at 28° C. (YLEX Expression Kit, Yeastearn Biotech, Cat. no.: FYY201-1KT).

TABLE XIII
Plasmids and strains

| | | | Proteins expressed | | |
|---|---|---|---|---|---|
| Names | Parental strains | Vector 1 | RbcX | GroES | GroEL |
| PO1f_01 | Yarrowia | YLEX1 | — | — | — |
| | lipolytica | | | | |
| PO1f_02 | Yarrowia | CB07 | S. elongatus | E. coli | E. coli |
| | lipolytica | | | | |

Yarrowia lipolytica PO1f (ATCC ®MYA2613 ™)
Genotype: MATA ura3-302 leu2-270 xpr2-322 axp2-deltaNU49 XPR2::SUC2

The evaluation of the impact of the chaperones was carried out on a culture of strains PO1f_01 and PO1f_02 in synthetic medium without leucine at 28° C. for 72 h.

### 1.6. Construction of CHO Cells

To ensure easy and versatile handling and high-performance transfer of the “Chaperones” plug-in on the set of cells of higher eukaryotes, a fourth-generation lentiviral transduction system was selected. These lentiviral particles make it possible to transfer the plug-in equally in primary, immortalized or transformed cells from various species of higher eukaryotes such as human or murine cells, for example.

On plasmid pFPP56 (Table III), each promoter controlling the expression of the GroES and GroEL genes from CAS20 and CAS21 (Table II) was replaced with a compatible promoter according to the literature (Table XIV).

TABLE XIV
Expression cassettes

| Names | Promoters | Open reading frame | Terminator |
|---|---|---|---|
| CAS54 | CMV | RbcX | hBeta globin |
| CAS55 | hEF-1a | GroES | hPKG1 |
| CAS56 | hUBC | GroEL | hGAPDH |

The region including the open reading frame of RbcX and the two expression cassettes CAS55 and CAS56 below were amplified by PCR and XhoI-KpnI cloned in commercial plasmid pLVX-Puro (Clontech, Catalog no. 632164).

TABLE XV
Expression vector

| | Origin | Cassette | Cassette | Cassette | | Host |
|---|---|---|---|---|---|---|
| Names | type | 1 | 2 | 3 | Marker | vector |
| pCB10 | Integrative | CAS54 | CAS55 | CAS56 | Puro | pLVX-Puro |

Plasmids pLVX-Puro or pCB10 were transformed using the Lenti-X Packaging System (Clontech) in Lenti-X 293T cells (Clontech) according to the kit's protocol. The supernatant containing the viral particles was filtered and added at 1/5 or 1/2 dilution to CHO cells cultured in 10 cm Petri dishes for a final volume of medium of 5 ml.

After 24 h of transduction, the cells are washed with PBS and fresh culture medium supplemented with 2 μg/ml puromycin is added for selection over 48 h at 37° C.

The cell line thus established is maintained under a concentration of 0.5 μg/ml puromycin in the culture medium.

TABLE XVI
Plasmid and strains

| | | | Proteins expressed | | |
|---|---|---|---|---|---|
| Names | Parental strains | Vector 1 | RbcX | GroES | GroEL |
| CHO-01 | CHO | pLVX-Puro | — | — | — |
| CHO-02 | CHO | pCB10 | S. elongatus | E. coli | E. coli |

### 1.7. Methods

### Culture Method 1: Growth on Glucose

The transformed cells are grown at 30° C. in ambient air on YNB medium (yeast without nitrogen base) supplemented with 6.7 g/l ammonium sulfate, 20 g/l glucose, 20 g/l agar for the agars supplemented with commercial CSM medium (MP Biomedicals) suited to the selection markers of the plasmids used for the transformation (-ura, -leu, -trp) and in the presence of 2μ/ml doxycycline. The cultures are stopped by cooling at 4° C. a generation before the end of the exponential phase. The culture is centrifuged, then spheroplasts are prepared by enzymatic digestion of cell walls with a zymolyase-cytohelicase mixture in hypertonic sorbitol medium (1.2 M sorbitol). The spheroplasts are washed in hypertonic sorbitol medium in the presence of saturating concentrations of PMSF and EDTA (protease inhibitors), then broken by repeated pipetting and mild sonication in isotonic sorbitol medium (0.6 M). After centrifugation at low speed (1500 rpm) to eliminate large debris then at moderate speed (4000 rpm) to collect debris of intermediate sizes and mitochondria, the supernatant is collected and the protein concentration is quantified by the Bradford method.

### Test for RuBisCO Activity In Vitro

In vitro, 15 μg of protein sample, from this cellular lysis, is added to the synthetic molecule RuBP (2 mM final) in suitable buffer (50 mM Tris/HCl pH 7.4, 10 mM MgCl₂⁺, 60 mM sodium bicarbonate), for a reaction volume of 200 μl, enabling the RuBisCO complex, expressed in the yeast lysate, to catalyze the formation of phosphoglyceric acid molecules. At variable times, the reactions are stopped by adding HCl (12.1 M) and the reaction products are analyzed by HPLC/MS in order to evaluate the carboxylase activity of the protein sample by assaying the phosphoglyceric acid produced over time.

### Cultures in Controlled Medium

Precultures were prepared on chemically defined medium. After thawing, 1 ml of a stock tube (−80° C.) was taken to inoculate a penicillin bottle (100 ml) containing 10 ml of culture medium, incubated for 18 hours at 30° C. and 120 rpm. The precultures were prepared in anaerobiosis (bottles previously flushed with nitrogen) and in the presence of doxycycline in order to avoid the toxicity problems observed in the presence of the PRK gene. The precultures were then washed three times (centrifugation, resuspension, vortex for 15 s) with physiological saline (NaCl, 9 g/l), then the cell pellet was resuspended in culture medium without doxycycline.

These cells stemming from the precultures were then inoculated in order to reach an initial optical density of 0.05 (or 0.1 g/1). The starting culture volume was 50 ml in aerobiosis (250 ml baffled Erlenmeyer flasks) or 35 ml in anaerobiosis (100 ml penicillin bottles).

The cultures were stopped after all glucose was consumed or ethanol production stopped. Each culture was prepared in triplicate.

### Analyses: Characterization of Extracellular Metabolites

The concentrations of glucose, formic acid and principal metabolites (ethanol; glycerol; acetic, succinic and pyruvic acids) were measured by high-performance liquid chromatography (HPLC). The apparatus used was a chromatograph (Waters, Alliance 2690) equipped with an Aminex HPX 87-H⁺ (300 mm×7.8 mm) column. Detection of the molecules was provided by a refractive index detector (Waters 2414 refractometer). The eluent was 8 mM H₂SO₄ at a flow rate of 0.5 ml/min, and the column temperature set at 50° C. In anaerobiosis, this analysis was carried out on a single bottle of each strain. In this case, the calculation of the standard deviation was carried out on the loss of mass, then applied to the metabolites.

### Example 2: Effect of the Combination of Chaperones on Reconstruction of the Carboxylase Activity of Type I RuBisCO in Yeast

The Calvin cycle enables plants and cyanobacteria to produce glucose from carbon dioxide. The critical step is the fixing of CO₂ on ribulose-1,5-bisphosphate (RuBP), a molecule having five carbons. This step requires an enzyme called RuBisCO (for ribulose-1,5-bisphosphate carboxylase/oxygenase). This enzyme enables the formation of an unstable six-carbon molecule which quickly gives two three-carbon 3-phosphoglycerate molecules. Several forms of RuBisCO exist. Form I consists of two types of subunits: large subunits (RbcL) and small subunits (RbcS), whose correct assembly further requires the intervention of at least one specific chaperone: RbcX. RuBP, the substrate of RuBisCO, is formed by reaction of ribulose-5-phosphate with ATP; this reaction is catalyzed by a phosphoribulokinase (PRK).

In this example, an artificial Calvin cycle is reconstituted by co-transformation of yeast strain CEN.PK 1605 by the combinations of vectors no. 3 and 4 of Table IV above, which enable the simultaneous expression of the RbcS and RbcL subunits of RuBisCO, the specific chaperone RbcX and the PRK enzyme from Synechococcus elongatus, with (combination 3 and 101) or without (combination 4) the general chaperones GroEL and GroES from E. coli or Synechococcus according to the combination number.

Thus, tests for RuBisCO activity in three independent experiments (A, B and C) are carried out from protein extracts from yeast cultures on glucose (protocol detailed above, point 1.3); their yield is evaluated by measuring phosphoglyceric acid production as a function of time. The results are presented in Table XVII below.

The experiment A shows that the presence of the chaperone RbcX alone, nevertheless specific to the RuBisCO complex, is not sufficient to enable the expression of an active enzymatic complex. Only the combination of both general chaperones GroES and GroEL from E. coli, associated in a stoichiometry suited to the presence of RbcX, makes it possible to detect increasing phosphoglyceric acid production over time.

Moreover, experiment C shows that RuBisCO activity drops dramatically by more than 90% when the RbcL/RbcS subunits from Synechococcus elongatus are associated with the homologous chaperones RbcX, GroES, GroEL2 from the same organism. This vividly illustrates the advantage of an association of heterologous chaperonins.

This example makes it possible to determine clearly that the association of heterologous bacterial general chaperones with the bacterial specialist chaperone RbcX is necessary to optimize the activity of a synthetic RuBisCO complex in yeast.

### Example 3: Protective Effect of the Combination of Chaperones Against the Toxicity of Recombinant Proteins

### Effect on Removing Toxicity Related to Ribulokinase Expression In Vivo, Independently of RuBisCO

The methods and analyses implemented are described in Example 1 above.

Expression of the only ribulokinase in yeast (strain 18b) involves a long latency phase (of more than 50 hours) and a drastic drop in its maximum growth rate (of 70% in aerobiosis and 82% in anaerobiosis) compared to the wild strain (WT) (Table XVIII).

This toxicity, induced by PRK, can be partially removed by co-expression in strain 102 of the chaperones GroES/GroEL from E. coli (removal of toxicity on growth rate of 26% in anaerobiosis and 42% in aerobiosis) or the chaperone RbcX from Synechococcus elongatus in strain 14b (removal of toxicity on growth rate of 34% anaerobiosis and 10% in aerobiosis).

Co-expression in strain 15 of the chaperones GroES/GroEL from E. coli and RbcX from Synechococcus elongatus makes it possible to restore the near totality of growth (removal of toxicity on growth rate of 78% in anaerobiosis and 63% in aerobiosis). This co-expression also makes it possible to completely eliminate the long latency phase.

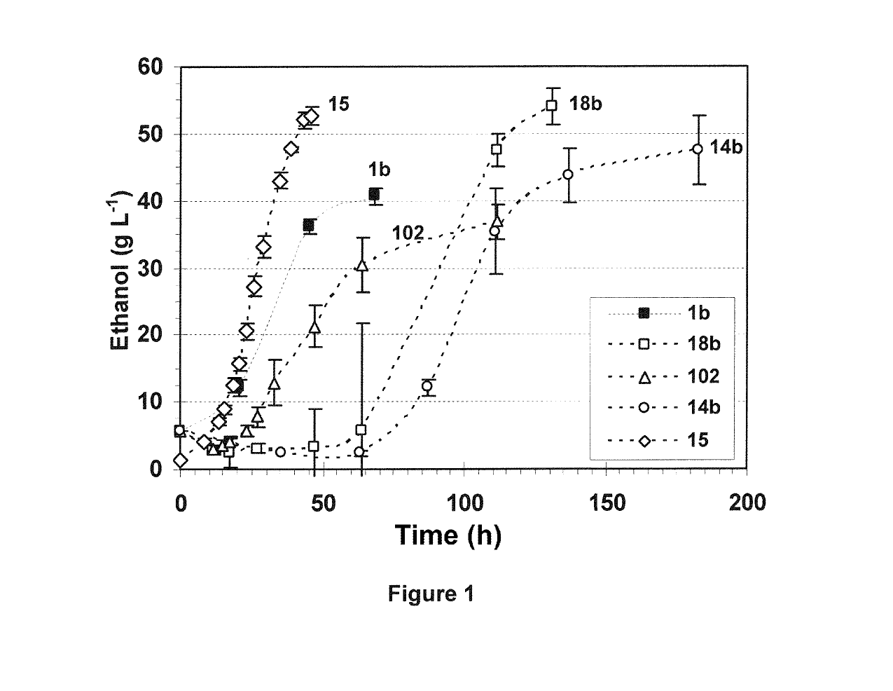

Toxicity related to the expression of the ribulokinase (PRK) affects alcohol fermentation characterized by a drop in ethanol productivity (strain 18b) (FIG. 1) directly related to the presence of the latency phase and to the drop in growth rate. Expression of the “CHAPERONES” engineering (GroES+GroEL from E. coli+RbcX from Synechococcus elongatus) not only makes it possible to completely remove the toxicity of the ribulokinase (PRK) and more particularly the accumulation of the product of the PRK-catalyzed reaction, but also consequently to increase ethanol productivity (strain 15), whereas the general chaperonin pair GroES/GroEL has only a partial effect (strain 102) and the expression of RbcX alone has none (strain 14b).

### Example 4: Exemplification of the General Effect on the Growth of a Transformed Cell

4.1. General Effect on the Growth of Saccharomyces cerevisiae in Fermentation

The methods and analyses implemented are described in Example 1 above. The results are presented in Table XIX below.

Advantageously, expression of the “CHAPERONES” engineering offers a proliferative advantage of 30% to strain 13b (RbcX+(Gros/GroEL) E. coli) compared to the control strain containing three “empty” plasmids (strain 1b), or strain 103 expressing only the E. coli general chaperones GroES/GroEL.

Moreover, the growth rate of strain 13b is equal to 96% and 86% of the growth rate of wild strain (WT) CEN.PK 113-7D not transformed and thus not stressed, in aerobiosis and anaerobiosis respectively (Table XVIII).

4.2. General Effect on the Growth of Pichia pastoris in Fermentation

The evaluation of the impact of the chaperones was carried out on a culture of strains PPGC115_01 and PPGC115_02 on minimal medium with glycerol at 30° C. for 14 h and induction with 1% methanol for 48 h to 100 h according to the protocol described in F. Wang et al. 2015 (PLoS One. 2015 Mar. 17; 10(3):e0120458. “High-level expression of endo-β-N-acetylglucosaminidase H from Streptomyces plicatus in Pichia pastoris and its application for the deglycosylation of glycoproteins.”).

The two strains were inoculated at the same cellular concentration evaluated on a fermentation of 100 h, the maximum mu of the strain calculated on the exponential phase of the growth curve has for strain PPGC115_02 a proliferative advantage on the order of 30% in relation to that of the control strain PPGC115_01.

4.3. General Effect on the Growth of Yarrowia lipolytica in Fermentation

Strains PO1f_01 and PO1f_02 were evaluated according to the protocol described in J M Nicaud et al. 2002 (Protein expression and secretion in the yeast Yarrowia lipolytica. FEMS Yeast Res. 2002 August; 2(3):371-9). The phenotype shows increased growth for the strain expressing the combination of chaperones. The proliferative advantage is evaluated at more than 35%.

### 4.4. General Effect on the Growth of CHO Cells

Lines CHO-01 and CHO-02 are inoculated at the same density (2×10⁶ cells per 10 cm dish) and growth is evaluated over 4 days. The cells are detached and individualized by treatment with trypsin and counted each day using an automatic counter. The growth rate of cells of line CHO-02 is on the order of 25% higher than that of the control line CHO-01. The combination of chaperones has an effect on cell doubling time.

These studies show that this “CHAPERONES” engineering makes it possible (i) to restore normal growth of eukaryotic cells, containing another engineering or not, but subjected to physicochemical stresses and (ii) to offer a proliferative advantage in relation to strains/cells used under the same conditions.

### Example 5: Exemplification of the Effect on the Production of Recombinant Proteins

5.1. Production of Human Growth Hormone in Saccharomyces cerevisiae The human growth hormone gene (GenBank: K02382.1) was synthesized and cloned downstream of the constitutive promoter TEF1 according to the cassette below.

TABLE XX
Expression cassette

| Names | Promoters | Open reading frame | Terminator |
|---|---|---|---|
| CAS54 | TEF1p | hGH | PGK |

TABLE XXI
Expression vectors

| | Origin | Cassette | Cassette | Cassette | | Host |
|---|---|---|---|---|---|---|
| Names | type | 1 | 2 | 3 | Marker | vector |
| pCB09 | 2μ | CAS54 | | | URA3 | PYeDP51 |
| pFPP56 | ARS415- | CAS19 | CAS21 | CAS22 | LEU2 | pFL36 |
| | CEN6 | | | | | |

These plasmids were transformed jointly in strain CEN.PK 1605 according to the transformation protocol previously described. The strains were selected on synthetic medium (-leu-ura).

TABLE XXII
Strains

| | Parental | | | Proteins expressed | | | |
|---|---|---|---|---|---|---|---|
| Names | strain | Vector 1 | Vector 2 | RbcX | GroES | GroEL | hGH |
| 200 | CEN. | PYeDP51 | pFPP56 | S. | E. coli | E. coli | |
| | PK1605 | | | elongatus | | | |
| 230 | CEN. | pCB09 | pFPP56 | S. | E. coli | E. coli | hGH |
| | PK1605 | | | elongatus | | | |
| 231 | CEN. | pCB09 | pFL36 | | | | hGH |
| | PK1605 | | | | | | |

Strains 200, 230 and 231 were grown in synthetic medium (-leu-ura) until an OD 600 nm of 0.7. The cells were collected and washed once with 1 ml of cold lysis buffer (1×PBS pH 7.4, 1 mM PMSF), then resuspended in 0.3 ml of Laemmli buffer and incubated for 5 min at 98° C. A serial dilution (1:10) is prepared and the same volume of each sample is deposited on an SDS-PAGE gel (gradient 4%-20%), transferred on a nitrocellulose membrane. hGH expression is evaluated with the antibody [GH-1] (ab9821, Abcam) and standardized in relation to expression of the ubiquitous gene GAPDH (ab9485, Abcam). Furthermore, hGH expression is quantified by ELISA with and according to the protocol of the Growth Hormone ELISA Kit, Human (Thermo Scientific, catalog no.: EHGH1).

The quantity of hGH protein produced evaluated in strain 230 is 40% higher than that obtained in strain 211.

5.2. Evaluation of Endogenous Luciferase Activity in Saccharomyces cerevisiae

The firefly luciferase gene was amplified from vector pGL4 (Promega) and cloned downstream of the constitutive promoter TEF1 according to the cassette below.

TABLE XXIII
Expression cassette

| Names | Promoters | Open reading frame | Terminator |
|---|---|---|---|
| CAS53 | TEF1p | fLuc | PGK |

TABLE XXIV
Expression vectors

| | Origin | Cassette | Cassette | Cassette | | Host |
|---|---|---|---|---|---|---|
| Names | type | 1 | 2 | 3 | Marker | vector |
| pCB08 | 2μ | CAS53 | | | URA3 | PYeDP51 |
| pFPP56 | ARS415- | CAS19 | CAS21 | CAS22 | LEU2 | pFL36 |
| | CEN6 | | | | | |

These plasmids were transformed jointly in strain CEN.PK 1605 according to the transformation protocol described previously. The strains were selected on synthetic medium (-leu-ura).

TABLE XXV
Plasmids and strains

| | | | | Proteins expressed | | | |
|---|---|---|---|---|---|---|---|
| Names | Parental strain | Vector 1 | Vector 2 | RbcX | GroES | GroEL | fLUC |
| 200 | CEN. PK1605 | PYeDP51 | pFPP56 | S. elongatus | E. coli | E. coli | |
| 210 | CEN. PK1605 | pCB08 | pFPP56 | S. elongatus | E. coli | E. coli | fLuc |
| 211 | CEN. PK1605 | pCB08 | pFL36 | | | | fLuc |

Strains 200, 210 and 211 were grown in synthetic medium (-leu-ura) until an OD 600 nm of 0.7. The cells were collected and washed once with 1 ml of cold lysis buffer (1×PBS pH 7.4, 1 mM PMSF), then resuspended in 0.3 ml of the same buffer. The suspended cells were lysed with glass beads (Fast Prep).

The concentration of the crude lysates was determined by the Bradford method (BioRad) and diluted to 0.5 mg/ml, and luciferase activities were determined using 5 μl of lysate per sample using the Luciferase Assay System (Promega) and luminescence evaluated on a luminometer.

The activity is standardized in relation to the quantity of total protein.

The luciferase activity evaluated in strain 210 is 60% higher than that evaluated in strain 211.

5.3. Evaluation of the Activity of a Recombinant Cellulase in Saccharomyces cerevisiae

The Chaperones engineering is associated with an engineering for expressing a cellulase, cellobiohydrolase 1 (CBH1) from Talaromyces emersonii (GenBank accession no. AAL89553) under promoter TEF2. The analysis of cellulase activity was carried out as described in Y. Ito et al. 2015 (Combinatorial Screening for Transgenic Yeasts with High Cellulase Activities in Combination with a Tunable Expression System. PLoS One. 2015 Dec. 21; 10(12)).

The activity recorded for the strain co-expressing the Cellulase engineering in the presence of Chaperones has an activity yield 37% higher than the strain expressing only the Cellulase engineering alone.

### 5.4. Improvement in Protein Production

The use of chaperones to improve protein production described previously for Saccharomyces can easily be implemented in any eukaryotic cell of interest and in particular in Pichia and Yarrowia, so as to optimize thereby the yield and/or the activity of endogenous or heterogeneous proteins. The person skilled in the art can in particular refer to the publications below to make a yeast expressing the combination of chaperones according to the invention produce various proteins of interest for the food-processing industry, the pharmaceutical field, biomass hydrolysis, energy, etc.:
Spohner S C, Müller H, Quitmann H, Czermak P. Expression of enzymes for the usage in food and feed industry with Pichia pastoris. J Biotechnol. 2015 May 20; 202:118-34.
Kim H, Yoo Si, Kang H A. Yeast synthetic biology for the production of recombinant therapeutic proteins. FEMS Yeast Res. 2014 Aug. 12.
Ahmad M, Hirz M, Pichler H, Schwab H. Protein expression in Pichia pastoris: recent achievements and perspectives for heterologous protein production. Appl Microbiol Biotechnol. 2014 June; 98(12):5301-17. doi: 10.1007/s00253-014-5732-5. Epub 2014 Apr. 18. Review.
Weinacker D, Rabert C, Zepeda A B, Figueroa C A, Pessoa A, Farias J G. Applications of recombinant Pichia pastoris in the healthcare industry. Braz J Microbiol. 2014 Mar. 10; 44(4):1043-8
Rabert C, Weinacker D, Pessoa A Jr, Farias J G. Recombinants proteins for industrial uses: utilization of Pichia pastoris expression system. Braz J Microbiol. 2013 Oct. 30; 44(2):351-6.
Spadiut O, Capone S, Krainer F, Glieder A, Herwig C. Microbials for the production of monoclonal antibodies and antibody fragments. Trends Biotechnol. 2014 January; 32(1):54-60.
Espejo-Mojica Á J, Alméciga-Díaz C J, Rodriguez A, Mosquera Á, Diaz D, Beltrán L, Díaz S, Pimentel N, Moreno J, Sánchez J, Sánchez O F, Córdoba H, Poutou-Piñales R A, Barrera L A. Human recombinant lysosomal enzymes produced in microorganisms. Mol Genet Metab. 2015 September-October; 116(1-2):13-23.
Gündüz Ergün B, Çalik P. Lignocellulose degrading extremozymes produced by Pichia pastoris: current status and future prospects. Bioprocess Biosyst Eng. 2015 Oct. 23.
Ledesma-Amaro R, Dulermo T, Nicaud J M. Engineering Yarrowia lipolytica to produce biodiesel from raw starch. Biotechnol Biofuels. 2015 Sep. 15; 8:148.
Kalyani D, Tiwari M K, Li J, Kim S C, Kalia V C, Kang Y C, Lee J K. A highly efficient recombinant laccase from the yeast Yarrowia lipolytica and its application in the hydrolysis of biomass. PLoS One. 2015 Mar. 17; 10(3):e0120156.
Zinjarde S S. Food-related applications of Yarrowia lipolytica. Food Chem. 2014; 152:1-10.
Hughes S R, Lopez-Núñez J C, Jones M A, Moser B R, Cox E J, Lindquist M, Galindo-Leva L A, Riaño-Herrera N M, Rodriguez-Valencia N, Gast F, Cedeño D L, Tasaki K, Brown R C, Darzins A, Brunner L. Sustainable conversion of coffee and other crop wastes to biofuels and bioproducts using coupled biochemical and thermochemical processes in a multi-stage biorefinery concept. Appl Microbiol Biotechnol. 2014 October; 98(20):8413-31.
Groenewald M, Boekhout T, Neuvéglise C, Gaillardin C, van Dijck P W, Wyss M. Yarrowia lipolytica: safety assessment of an oleaginous yeast with a great industrial potential. Crit Rev Microbiol. 2014 August; 40(3):187-206.
Celik E, Calik P. Production of recombinant proteins by yeast cells. Biotechnol Adv. 2012 September-October; 30(5):1108-18.
Sabirova J S, Haddouche R, Van Bogaert I, Mulaa F, Verstraete W, Timmis K, Schmidt-Dannert C, Nicaud J, Soetaert W. The ‘Lipo Yeasts’ project: using the oleaginous yeast Yarrowia lipolytica in combination with specific bacterial genes for the bioconversion of lipids, fats and oils into high-value products. Microb Biotechnol. 2011 January; 4(1):47-54.

1) A transformed eukaryotic cell comprising:
(i) an expression cassette containing a sequence encoding a chaperone RbcX involved in the folding of a bacterial form I Rubisco enzyme, under the transcriptional control of a suitable promoter;
(ii) an expression cassette containing a sequence encoding a bacterial general chaperone GroES, under the transcriptional control of a suitable promoter; and
(iii) an expression cassette containing a sequence encoding a bacterial general chaperone GroEL, under the transcriptional control of a suitable promoter.

2) The eukaryotic cell according to claim 1, wherein the chaperone RbcX is a cyanobacterial chaperone.

3) The eukaryotic cell according to claim 1, wherein at least one of the general chaperones GroES and GroEL comes neither from a cyanobacterium nor from another bacterium expressing a RuBisCO complex.

4) The eukaryotic cell according to claim 1, wherein the three expression cassettes form a continuous block of genetic information.

5) The eukaryotic cell according to claim 1, wherein the expression cassettes of the three chaperones are carried by a single episomal genetic element.

6) The eukaryotic cell according to claim 1, wherein it further comprising at least one expression cassette for a heterologous protein other than said chaperones, or in that it has undergone a sequence engineering modifying the level of expression and/or the sequence of an endogenous protein.

7) The eukaryotic cell according to claim 1, that does not contain the sequence encoding an RbcL or RbcS subunit of a bacterial form I RuBisCO enzyme.

8) The eukaryotic cell according to claim 1, that is a yeast.

9) A method for improving the physiology of a eukaryotic cell, said method comprising combining expression cassettes enabling the expression of a chaperone RbcX and the chaperones GroES and GroEL.

10) The method according to claim 9, wherein said eukaryotic cell is a yeast.

11) The method according to claim 9 wherein said eukaryotic cell containing at least one expression cassette for a heterologous protein other than said chaperones, or having undergone a sequence engineering modifying the level of expression and/or the sequence of an endogenous protein.

12) The method according to claim 8 that increases the growth rate of said eukaryotic cell.

13) The method according to claim 8 that increases the resistance of said eukaryotic cell to an environmental stress.

14) The method according to claim 13, wherein the environmental stress is due to the toxicity of an element present in the culture medium of the eukaryotic cell.

15) The method according to claim 9 that increases the resistance of said cell to the toxicity of a compound synthesized by the eukaryotic cell.

16) The method for producing a recombinant protein using a eukaryotic cell according to claim 1.

17) A biotechnological process for producing at least one compound that is a chemical molecule or a protein, said process comprising a step of culturing a eukaryotic cell according to claim 1 under conditions enabling the synthesis, by said eukaryotic cell, of said compound, and optionally a step of collecting said compound thus synthesized.

18) A process for producing a recombinant protein, the process comprising (i) inserting a sequence encoding said protein into a eukaryotic cell expressing RbcX, GroES and GroEL, (ii) culturing said cell under conditions enabling the expression of said sequence and optionally (iii) collecting or purifying said protein.

19) The process according to claim 17, wherein said protein is an enzyme.

20) The process according to claim 17, wherein said protein is a hormone.

21) The eukaryotic cell of claim 8, wherein the yeast is Saccharomyces cerevisiae, Yarrowia lipolytica, or Pichia pastoris.
